(19) United States
(12) Patent Application Publication
(10) Pub. No.: US 2021/0383902 A1
(43) Pub. Date: Dec. 9, 2021

(54) VERIFIABLE STD (SEXUALLY TRANSMITTED DISEASE) AND HEALTH STATUS VALIDATION SCANNING APPLICATION

(71) Applicant: Ricky Booker, Gilbert, AZ (US)

(72) Inventor: Ricky Booker, Gilbert, AZ (US)

(21) Appl. No.: 16/896,438

(22) Filed: Jun. 9, 2020

(51) Int. Cl. G16H 10/60; G16H 15/00; G16H 50/50; G16H 50/20; G16H 50/30; G16H 30/20; G16H 30/40

(52) U.S. Cl. CPC G16H 10/60; G16H 15/00; G16H 50/50; G16H 30/40; G16H 50/30; G16H 30/20; G16H 50/20

(57) ABSTRACT

A method of providing health status information, by an application, comprising a computer readable storage storing instructions, and a processor configured to perform the method, the method comprising:

The application and method of providing health status information. The application includes with information for others to review about any disease and medical history when checking for the Sexual partner. This application is for all type of genders, age group and allowing them the ability to verify and validate user's current sexual health status. Using the QR Scanner from one device to another device. All the data in the application of the users will be verified before uploading in the database for others to review.

### COPYRIGHT NOTICE

A portion of the disclosure of this patent document contains material which is subject to copyright protection. The copyright owner has no objection to the facsimile reproduction by anyone of the patent disclosure, as it appears in the Patent and Trademark Office patent files or records, but otherwise reserves all copyright rights whatsoever.

### BACKGROUND

### Field of the Invention

Provided is an application and method of providing health status information. The application includes: a storage configured to store a first image including a face of a user and first health status information entered by the user; with information for others to review about any disease and past medical history when checking for the Sexual partner. This application is for all type of genders, age group and allowing them the ability to verify and validate user's current sexual health status.

### Description of the Related Art

Recently, various electronic apparatuses provide health-related functions to users. For these apparatuses, various equipment is used to measure health-related information. In particular, special apparatuses are used to measure health-related information, for example, to photograph a subject by using ultrasonic waves, computer tomography (CT), or magnetic resonance imaging (MRI), to measure blood pressure by using a blood pressure gauge, or to measure a weight by using a scale. However, since it is difficult for ordinary users to easily use such special apparatuses, the ordinary users may have difficulty in obtaining heath status information.

Accordingly, health status information may be provided to the user without having to use a special apparatus if a health status of the user can be determined based on the image.

According to an aspect of an exemplary embodiment, there is provided a device including: a storage configured to store a first image including a face of a user and first health status information extracted from the first image; an imager configured to capture an image; a controller configured to control the imager to capture a second image including the face of the user and to extract second health status information from the captured second image; and a display configured to output the second image and information other than the stored first health status information from among the extracted second health status information. U.S. Pat. No. 10,366,487B2.

In particular, the RTLS can be applied to freight containers or the transportation field for physical distribution to check the moving route of the objects in real time and is useful in terms of the security and management of physical distribution.

The device may further include: a user interface unit configured to receive a user input for unlocking the device when in a locked state, wherein the controller may be configured, when the user input for unlocking the device in the locked state is received, to control the imager to capture the second image and to extract the second health status information from the captured second image.

A kind of health status real time monitor and diagnosis and treatment commending system, by human body biological characteristics sensor group, wireless network, personal server, high in the clouds medical server, medical resource supplier composition, the structure of human body biological characteristics sensor group is made up of processing module, sensor assembly and radio receiving transmitting module and power supply module. The present invention provides the user medical resource recommendation list based on QoE. This function is based on QoE and user can be helped to select most suitable professional medical mechanism, and patient can avoid blindly seeking medical advice. Health care provider can get the health status record of patient, and these records can help doctor to make patient to diagnose the most accurately.

The present invention devises one and utilizes biosensor and Internet service for user based on user experience quality (QoE) The scheme providing medical resource to recommend, belongs to public medical and health and public service technical field. It is applicable to build public health Multiple fields such as status monitoring, health and epidemic prevention and raising Consumer's Experience.

In busy daily life, people have no time to attend to the health status of oneself, until sick time just find oneself body has the worst. When an ill, he both do not known sick which kind of, also do not know how tackle the present situation, only One can do is exactly to go to see a doctor in time. But for some disease, it had been necessary for obtaining timely before hospital Reply processes, such as heart disease and stroke. Additionally, they do not know that the treatment of this disease is good at by which hospital yet, which is not arrogated to oneself Long Expense outside which hospital's medical insurance they can undertake, which cannot. Enough there is reference value not grasping. The when of medical treatment resource information, what they can do is only the hospital admission purposelessly arriving periphery, when this is to waste very much in fact Between.

Provide the embodiment for system, computer media and the computer implemented method state of driver being monitored when driver driving. A kind of system including one group of sensors, this group sensor is configured to be arranged in vehicle gather driver's status data. This system processes driver's status data to determine that driver is the most experiencing health status or Health risk and determining whether the body gesture of driver meets human engineering. The healthiness alarm representing this health status is produced in response to health status. Experience Health risk in response to driver and produce the healthiness alarm the quiescing vehicle that represent this Health risk. Body gesture in response to driver do not meet human engineering and identify and provide for driver physical posture need perform so that driver is positioned at the regulation of acceptable body gesture on human engineering.

When driving vehicle, (such as, driver faces the contingency being involved in other vehicle Collide) risk. When driver engenders can result in of such as tired or sleep deprivation etc During the health status of car accident, such risk can increase. Unfortunately, car is driven Risk be not limited to car accident. Such as, when driving vehicle, body gesture is bad Driver can increase oneself suffer from such as cervical pain, low back pain, sciatic nerve problem it. The risk of the physical health issues of class. Therefore, driver can face kinds of risks when driving vehicle.

A kind of health status real time monitor and diagnosis and treatment commending system, by human body biological characteristics sensor group, wireless network, personal server, high in the clouds medical server, medical resource supplier composition, the structure of human body biological characteristics sensor group by Processing module, sensor assembly and radio receiving transmitting module and power supply module composition.

Health care provider is automatically collected various data that user uploads and stores, and data integration to user's medical treatment is remembered. In the case of user goes to hospital or is in other necessity, with reference to the history health status of user, in conjunction with this user Up-to-date information and other known relevant user profile provide the user appropriate guidance.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a sexual health status of the partner so that you choose your partner by looking at the medical condition of the partner.

The present invention has been made to solve the mentioned problems, and it is an object of the present invention to let one decide if the partner has no specific disease and they are safe when choosing the right partner. The application scans the QR code of the second person and let you validate health status.

It is also the primary objective of the invention to let the application scan other partners through QR Scanner and provide complete health report which will help the other partner in choosing the right partner.

In this application one user with import the medical tests/report and build up a profile. After the results are verified by the software the applicant status of medical condition will be available for others to review when someone else scan them for data.

It is another objective of the invention to provide service related to booking the appointment with the doctor, booking the medical tests.

A further object is to provide a system between the user and doctor to consult with each other through the application in future.

A still further object is to propose a system that ensures transparency and trust in dynamic marketplace.

It is a further object of the present invention to provide a new and improved method which is of a reliable system.

This application is for all the age group, targeting all type of peoples who are interested in dating someone and want to get information for health safety.

Any object of any type may be brought into the platform in a simplified, single app and screen workflow.

This Summary is provided merely for purposes of summarizing some example embodiments, so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### DETAILED DESCRIPTION

Detailed descriptions of the preferred embodiment are provided herein. It is to be understood, however, that the present invention may be embodied in various forms. Therefore, specific details disclosed herein are not to be interpreted as limiting, but rather as a basis for the claims and as a representative basis for teaching one skilled in the art to employ the present invention in virtually any appropriately detailed system, structure or manner.

One aspect of the present application is directed to provide an automated QR scan code Realtime verification of the partner health status.

The application is as soon the user logins; the application will share the brand logos and name. And after that the user will make a profile in the application. The sign-up procedure will have two step verification.

After profile setup the dashboard of the application will be having scanner to scan with other users.

When one user scans the other users profile the alert will be shown on the receive end that do, they want to show the reports to other users. Only after giving the permission the sender can review the reports of the receiver.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

1. A system and procedure comprising of:
A memory configured to store user information,
a QR Scanner;
Displays the required information on output screen
Determining the health status of the user
